(19) United States
(12) Patent Application Publication
(10) Pub. No.: US 2006/0229558 A1
(43) Pub. Date: Oct. 12, 2006

(54) Medical infuser device

(75) Inventors: Brian K. Heston, Dover, OH (US); Mark R. Rufener, Dover, OH (US)

Correspondence Address: ZIMMER TECHNOLOGY - ROBERTS, P.O. BOX 1268, ALEDO, TX, 76008, US

(73) Assignee: Zimmer Technology, Inc.

(21) Appl. No.: 11/104,975

(22) Filed: Apr. 12, 2005

(51) Int. Cl. A61M 37/00

(52) U.S. Cl. 604/131

(57) ABSTRACT

An elastomeric infusion pump apparatus includes an elastomeric bladder and an axially-variable core positioned within the bladder. The bladder and the core are co-operable between a first state in which the bladder has expanded the core and a second state in which the core stretches the bladder. An apparatus for infusing a pharmaceutically active liquid supplied by an external source includes an elastomeric bladder and an axially-variable core positioned within the bladder. The bladder and the core are co-operable through an infusion cycle during which the bladder axially expands the core as the liquid is forced into the bladder from the external source, the bladder forces the liquid through the core after the liquid has been forced into the bladder, and the core radially stretches the bladder after the bladder has forced the liquid through the core.

### FIELD OF THE INVENTION

The present invention relates generally to the field of pharmaceutical liquid infusion devices, and, more particularly, to an elastomeric infusion pump apparatus.

### BACKGROUND

It is often desirable to supply ambulatory medical patients with pharmaceutically active liquids over extended time periods at controlled rates. Numerous devices for doing so have included elastomeric bladders designed to (when filled) exert pressures to pump such liquids to patients. For example, historical devices (including exemplary manners of operation and exemplary materials for their construction) are disclosed in U.S. Pat. No. 6,413,239 to Burns et al., U.S. Pat. No. 4,915,693 to Hessel, and U.S. Pat. No. 4,769,008 to Hessel, which are all expressly incorporated herein by reference for all purposes.

Problems encountered with elastomeric bladder type infusion equipment have included pumping pressure changes as their bladders have progressively emptied during their infusion cycles, pumping pressure surges as their bladders have forcefully contracted at the terminal ends of their infusion cycles, and residual (wasted) medications trapped or otherwise left over in the devices after their bladders have reached the terminal ends of their infusion cycles.

Historically, devices designed to address the above-noted problems have been undesirably costly and/or complex.

### SUMMARY OF THE INVENTION

The present invention provides an elastomeric infusion pump apparatus including an elastomeric bladder and an axially-variable core positioned within the bladder. The bladder and the core are co-operable between a first state in which the bladder has expanded the core and a second state in which the core stretches the bladder.

The present invention provides an apparatus for infusing a pharmaceutically active liquid supplied by an external source. The apparatus includes an elastomeric bladder and an axially-variable core positioned within the bladder. The bladder and the core are co-operable through an infusion cycle during which the bladder axially expands the core as the liquid is forced into the bladder from the external source, the bladder forces the liquid through the core after the liquid has been forced into the bladder, and the core radially stretches the bladder after the bladder has forced the liquid through the core.

The present invention provides an apparatus for infusing a pharmaceutically active liquid supplied by an external source. The apparatus includes elastomeric means for pumping the liquid, and means, coupled to the elastomeric means and at least partially disposed within the elastomeric means, for controlling at least one operation of the elastomeric means.

The above-noted features and advantages of the present invention, as well as additional features and advantages, will be readily apparent to those skilled in the art upon reference to the following detailed description and the accompanying drawings, which include a disclosure of the best mode of making and using the invention presently contemplated.

### BRIEF DESCRIPTION OF THE DRAWINGS

FIG. 1 shows a perspective view of an exemplary medical infuser device apparatus according to the present invention;

FIG. 2 shows a partially exploded perspective view of the exemplary apparatus of FIG. 1;

FIG. 3 shows a partially exploded perspective view of the core, the bladder, and the O-rings of the exemplary apparatus of FIG. 1;

FIG. 4 shows a partially exploded perspective view of the forked members of the exemplary apparatus of FIG. 1;

FIG. 5 shows a fully exploded perspective view of the forked members of the exemplary apparatus of FIG. 1;

FIG. 6 shows a cross-sectional view of the exemplary apparatus of FIG. 1 (taken along line 6-6 of FIG. 1) in an exemplary first operational state;

FIG. 7 shows a cross-sectional view of the exemplary apparatus of FIG. 1 (taken along line 7-7 of FIG. 1) in an exemplary second operational state; and

FIG. 8 shows a fluid flow block diagram for exemplary operations of the exemplary apparatus of FIG. 1.

### DETAILED DESCRIPTION OF EXEMPLARY EMBODIMENT(S)

Like reference numerals refer to like parts throughout the following description, the accompanying drawings, and the claims.

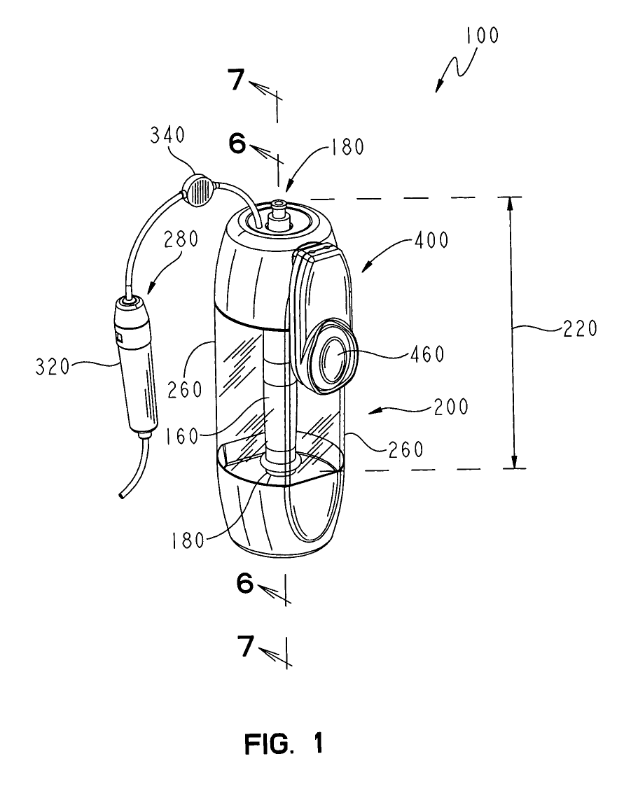

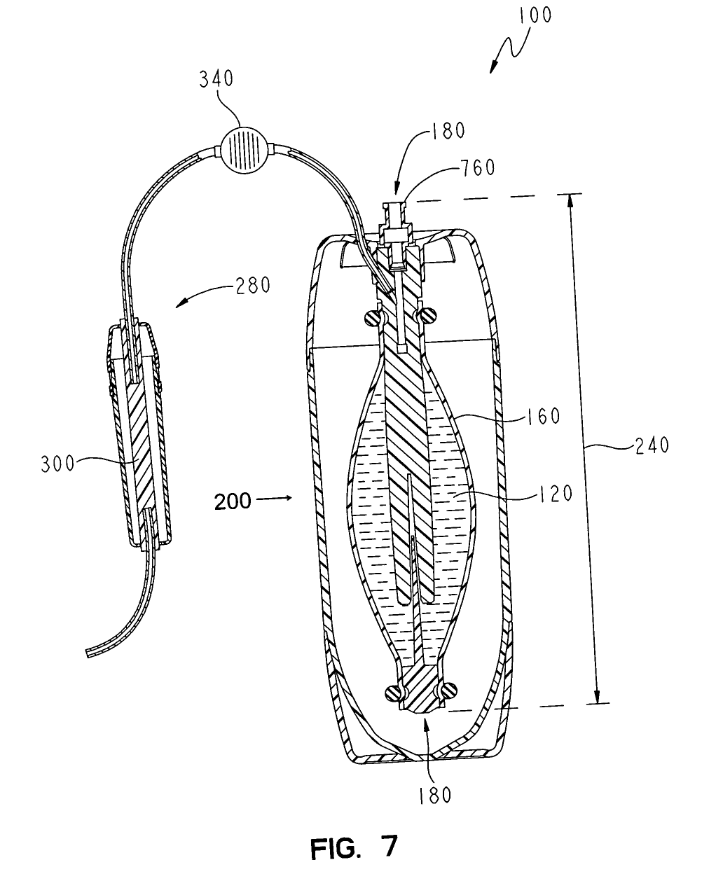

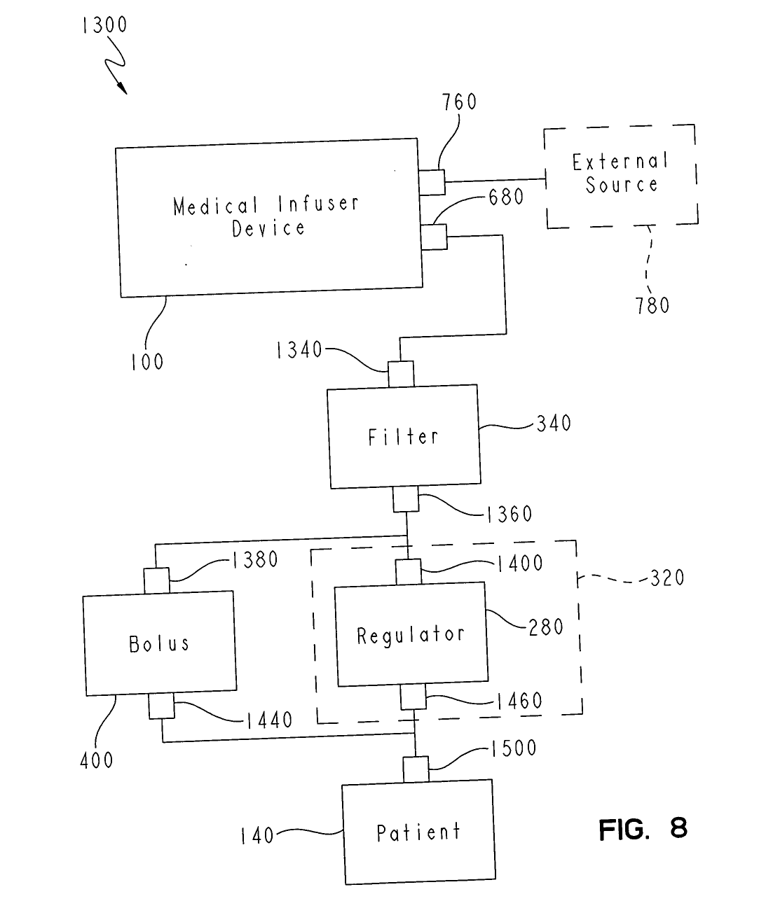

FIG. 1 shows a perspective view of an exemplary medical infuser device apparatus 100 according to the present invention. Among other things, apparatus 100 is configured to deliver a pharmaceutically active liquid 120 (see FIG. 7) to a patient 140 (see FIG. 8) at a generally constant flow rate. General principles of operation for such devices are well known. However, it should be appreciated from apparatus 100 that the present invention is, among other things, considerably less complex and easier to manufacture than historical devices.

Apparatus 100 includes a generally tubular elastomeric collapsing bladder 160 disposed concentrically about an axially-variable core 180 within a rigid outer casing or housing 200. As known in the art, bladder 160 is configured to exert suitable pressure on liquid 120 and is made from a suitable natural or synthetic rubber or any other suitable elastomeric composition that is suitably inert in the presence of liquid 120.

Core 180 is discussed further below. However, here it is noted that the term “axially-variable” as used herein is meant to indicate that core 180 is axially-expandable from a contracted state in which core 180 has a contracted longitudinal span 220 to an expanded state in which core 180 has an expanded longitudinal span 240 (see FIG. 7) and, conversely, that core 180 is axially-contractable from its expanded state to its contracted state.

Housing 200 is configured as known in the art to allow visual observation of bladder 160 while protecting bladder 160 from puncture risks and ultraviolet degradation. Accordingly, housing 200 includes a suitably transparent section 260. Section 260 is made from a polycarbonate, acrylic, or any other suitable ultraviolet blocking material.

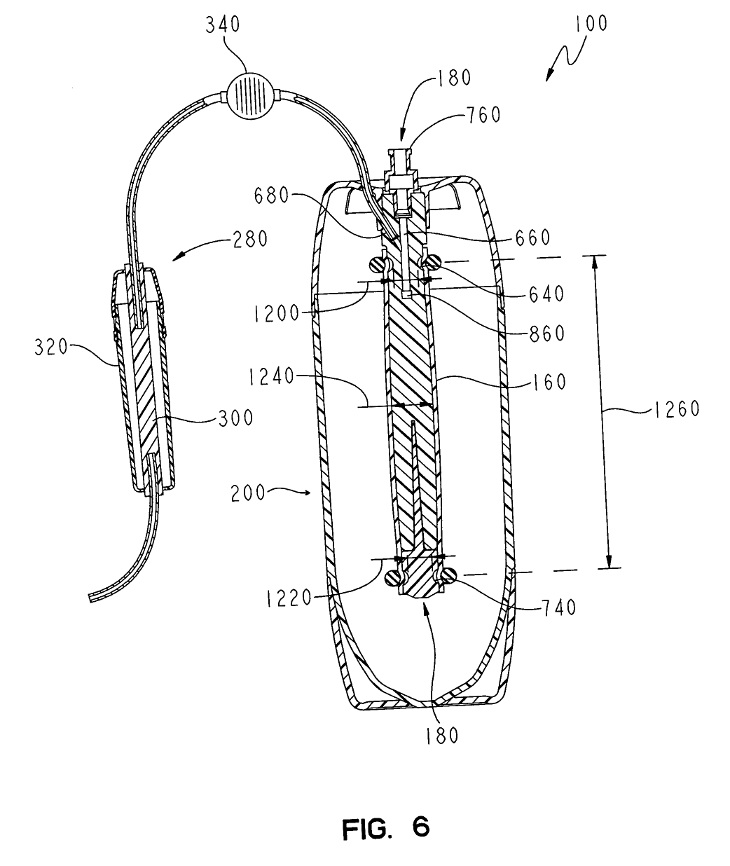

Apparatus 100 also includes an interchangeable regulator assembly 280. Assembly 280 includes a micro bore glass capillary 300 (see FIG. 6 and FIG. 7) that is supplied in differing flow configurations. Micro bore glass capillary 300 is configured as known to suitably regulate fluid 120 after it is expelled from bladder 160 through core 180 during operation. The flow rate through micro bore glass capillary 300 is low enough to become the path of greatest resistance such that micro bore glass capillary 300 regulates the effluent flow rate of apparatus 100. Assembly 280 further includes a tamper-resistant casing or housing 320 that encloses micro bore glass capillary 300 and cannot be readily disassembled after micro bore glass capillary 300 is enclosed therein.

Apparatus 100 also includes a filter assembly 340. Filter assembly 340 screens particulate/microbial matter ahead of interchangeable regulator assembly 280 to eliminate occlusions. Assembly 340 preferably has a flow rate so high that it does not significantly impact the overall flow rate of apparatus 100.

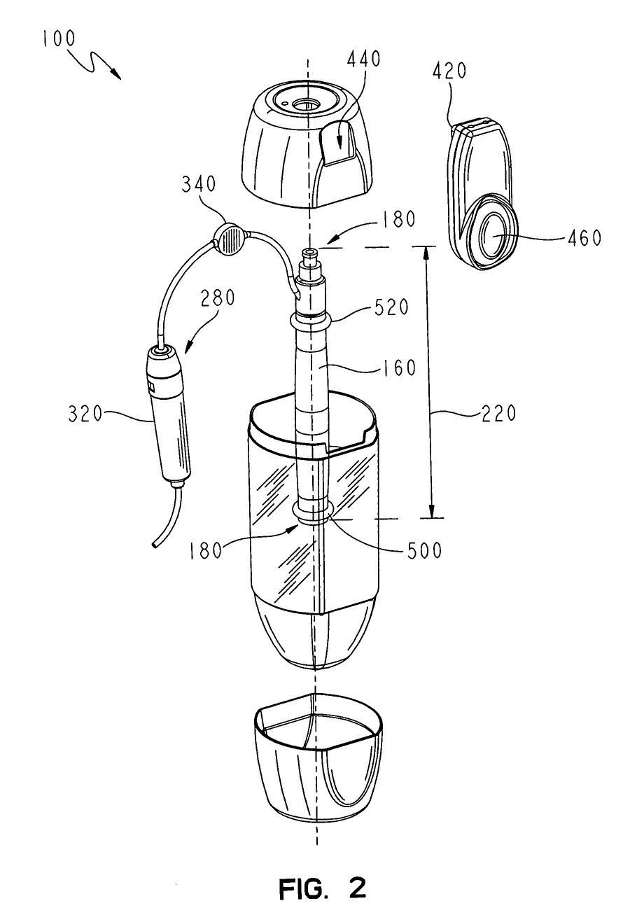

Apparatus 100 also includes a bolus mechanism 400. Mechanism 400 20 includes a tab or clip 420 (partially discernable in FIG. 2) that is inserted into a corresponding slot 440 (see FIG. 2) to attach mechanism 400 to housing 200, and mechanism 400 includes a bolus dose button 460. Mechanism 400 is hydro-mechanically configured as known to accumulate a controlled bolus dose of liquid 120 and to deliver the bolus dose (within limits) to patient 140 upon demand via actuation of button 460.

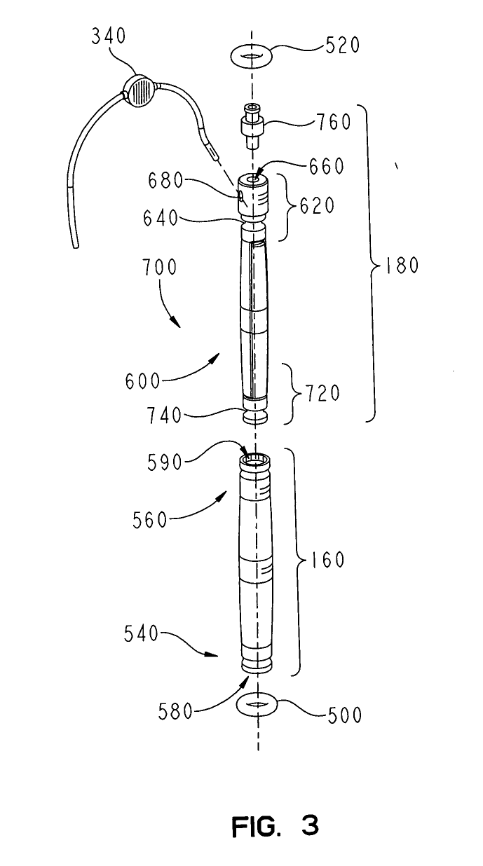

FIG. 2 shows a partially exploded perspective view of apparatus 100. As at least partially discernable in FIG. 2, apparatus 100 also includes an O-ring 500 and an O-ring 520 that help fluid-tightly seal an end 540 (see FIG. 3) of bladder 160 and an opposing end 560 (see FIG. 3) of bladder 160, respectively, to core 180.

FIG. 3 shows a partially exploded perspective view of core 180, bladder 160, O-ring 500, and O-ring 520. As at least partially discernable in FIG. 3, end 540 of bladder 160 defines an opening 580 and end 560 of bladder 160 defines an opposing opening 590. Meanwhile, core 180 includes a generally clothespin-shaped, elongated forked member 600. Member 600 is produced in accordance with suitable known thermoplastic forming techniques, and is made from acrylic, styrene, or any other rigid thermoplastic material that is suitably inert when immersed in liquid 120. Member 600 has an end portion 620 that defines an annular groove 640, a duct 660, and a duct 680. Additionally, core 180 includes a generally clothespin-shaped, elongated forked member 700 that is axially-slidably engaged with member 600 as discussed further below. Member 700 is preferably made from the same material(s) as member 600. Member 700 includes an end portion 720 that defines an annular groove 740. It should be appreciated that O-ring 500 presses end 540 of bladder 160 into groove 740 to fluid-tightly seal portion 720 of member 700 to bladder 160 proximal to opening 580, while O-ring 520 presses end 560 of bladder 160 into groove 640 to fluid-tightly seal portion 620 of member 600 to bladder 160 proximal to opening 590. Core 180 further includes a luer filling port 760 or any other suitable valve positioned in duct 660 for receiving a supply of liquid 120 from an external source 780 (see FIG. 8) as known in the art.

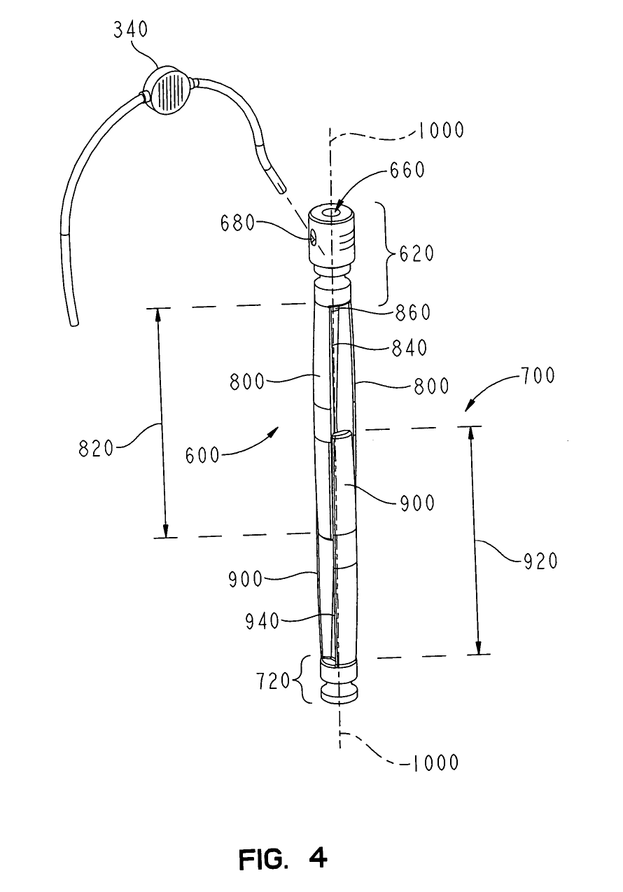

FIG. 4 shows a partially exploded perspective view of member 600 and member 700. As at least partially discernable in FIG. 4, member 600 includes a symmetrical pair of elongated branches 800 having a length 820. Member 600 further includes a relatively thin, flat crotch-like wall 840 extending between branches 800 from about the middle of their length towards portion 620. It is noted that wall 840 does not extend all the way to portion 620 and, thus, branches 800 and wall 840 define a generally rectangular gap or space 860. Space 860 allows easy entry of liquid 120 into apparatus 100 and equal ease of expulsion. Similarly to member 600, member 700 includes a symmetrical pair of elongated branches 900 having a length 920. Further, member 700 includes a relatively thin, flat crotch-like wall 940 extending between branches 900 from about the middle of their length all the way to portion 720. Branches 800 slidably fit between branches 940 and vice-versa. Thus, the “axially-slidably” or “axially-slidable” engagement of member 600 and member 700 is at least partially discernable from FIG. 4. In other words, member 600 and member 700 are slidable on each other such that portion 620 is movable towards portion 720 generally along an axis 1000 and, conversely, member 600 and member 700 are slidable on each other such that portion 620 is movable away from portion 720 generally along axis 1000.

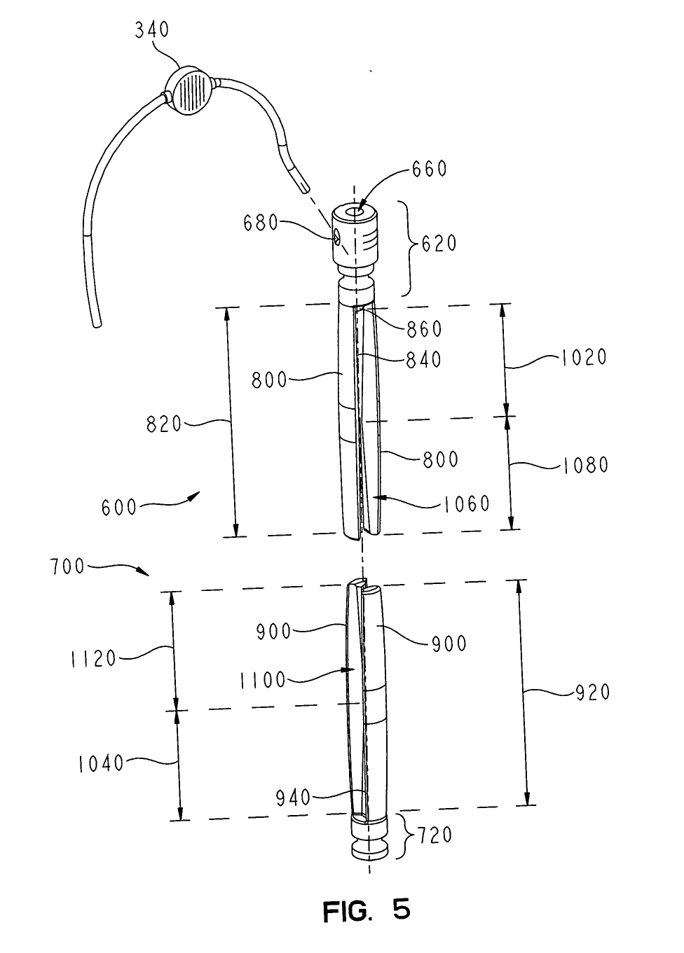

FIG. 5 shows a fully exploded perspective view of member 600 and member 700. As at least partially discernable in FIG. 5, wall 840 has a length 1020 that is about half the size of length 820, while wall 940 has a length 1040 that is about half the size of length 920. Also, it should be appreciated that branches 800 define a generally longitudinal space or slit 1060 therebetween. Slit 1060 has a length 1080 that is about the same as length 1020. Similarly, branches 900 define a generally longitudinal space or slit 1100 therebetween. Slit 1100 has a length 1120 that is about the same as length 1040. Additionally, it is noted that length 920 is about the same as the sum of length 1020 and length 1080.

FIG. 6 shows a cross-sectional view of apparatus 100 (taken along line 6-6 of FIG. 1) in an exemplary first operational state. More particularly, FIG. 6 shows a cross-sectional view of apparatus 100 when bladder 160 is practically empty and core 180 is fully contracted. As at least partially discernable in FIG. 6, duct 660 extends through portion 620 of member 600 (see also FIG. 3, FIG. 4, and FIG. 5) and opens at space 860. Further, duct 680 branches from duct 660 and thus, duct 680 communicates with space 860 as well. Additionally, groove 640 and groove 740 (see also FIG. 3) have a diameter 1200 and an identical diameter 1220, respectively. It is noted here that branches 800 and branches 900 have slightly arcuate profiles such that when core 180 is fully contracted, they give core 180—about mid way (longitudinally) between groove 640 and groove 740 (see also FIG. 3)—a maximum diametrical span 1240 that is greater than diameter 1200 (and thus, also equally greater than diameter 1220) by an amount sufficient to radially stretch bladder 160 at diameter 1240 by at lease 10% (preferably by about 12%); and it is also noted that core 180 is further configured to provide an axial distance 1260 between groove 640 and groove 740 that is sufficient to axially stretch bladder 160 by at lease 20% (preferably by about 25%).

FIG. 7 shows a cross-sectional view of apparatus 100 (taken along line 7-7 of FIG. 1) in an exemplary second operational state. More particularly, FIG. 7 shows a cross-sectional view of apparatus 100 after liquid 120 has been injected into bladder 160 through port 760 in a known manner, which has radially and axially expanded bladder 160 according to the exemplary embodiment (which, in turn, has axially expanded core 180 according to the exemplary embodiment).

Next, FIG. 8 shows a fluid flow block diagram 1300 for exemplary operations of apparatus 100. In operation of apparatus 100 through an exemplary infusion cycle beginning with bladder 160 empty and core 180 fully collapsed (see FIG. 6), external source 780 of liquid 120 (for example, a syringe or any other suitable source) is used in a known manner to introduce liquid 120 into bladder 160 through port 760 (see also FIG. 6). As liquid 120 is introduced into bladder 160, bladder 160 first begins to expand radially and then also begins to expand axially (depending on the initial stretch conditions, discussed above in connection with FIG. 6) as well as radially. As bladder 160 expands axially, bladder 160 in turn axially expands core 180 (see FIG. 7). After the desired amount of liquid 120 has been introduced such that bladder 160 has expanded and has in turn axially expanded core 180 (see FIG. 7), external source 780 is withdrawn.

As port 760 only operates as a one-way valve and bladder 160 exerts elastomeric pressure on liquid 120, after liquid 120 is introduced as discussed above bladder 160 operates to force liquid 120 from core 180 through duct 680. Further, in a known manner(s): duct 680 is coupled to a suitable inlet 1340 of filter 300; a suitable outlet 1360 of filter 300 is coupled to a suitable inlet 1380 of bolus 400 and coupled to a suitable inlet 1400 of chosen interchangeable flow regulator assembly 280; and a suitable outlet 1440 of bolus 400 and a suitable outlet 1460 of chosen interchangeable flow regulator assembly 280 are coupled (through a suitable catheter 1500 or other suitable fluid cannula device) to patient 140.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. An elastomeric infusion pump apparatus, comprising:
an elastomeric bladder; and
an axially-variable core positioned within the bladder;
wherein the bladder and the core are co-operable between a first state in which the bladder has expanded the core and a second state in which the core stretches the bladder.

2. The apparatus of claim 1, wherein the core includes a forked member and further includes a second member axially-slidably engaged with the forked member.

3. The apparatus of claim 2, wherein the bladder defines a first opening and the forked member includes an end portion fluid-tightly sealed to the bladder proximal to the first opening.

4. The apparatus of claim 3, wherein the bladder defines a second opening and the second member includes an end portion fluid-tightly sealed to the bladder proximal to the second opening.

5. The apparatus of claim 4, wherein the forked member further includes a pair of elongated branches extending from the end portion of the forked member, and the end portion of the forked member defines a duct including an opening positioned between the branches.

6. The apparatus of claim 5, wherein in the second state the core radially stretches the bladder by at least 10%.

7. The apparatus of claim 6, wherein in the second state the core axially stretches the bladder by at least 20%.

8. The apparatus of claim 6, wherein in the second state the core radially stretches the bladder by about 12%.

9. The apparatus of claim 8, wherein in the second state the core axially stretches the bladder by about 25%.

10. The apparatus of claim 5, wherein the second member further includes a pair of elongated branches extending from the end portion of the second member.

11. The apparatus of claim 10, wherein in the second state the core radially stretches the bladder by at least 10%.

12. The apparatus of claim 11, wherein in the second state the core axially stretches the bladder by at least 20%.

13. The apparatus of claim 11, wherein in the second state the core radially stretches the bladder by about 12%.

14. The apparatus of claim 13, wherein in the second state the core axially stretches the bladder by about 25%.

15. An apparatus for infusing a pharmaceutically active liquid supplied by an external source, the apparatus comprising:
an elastomeric bladder; and
an axially-variable core positioned within the bladder;
wherein the bladder and the core are co-operable through an infusion cycle during which the bladder axially expands the core as the liquid is forced into the bladder from the external source, the bladder forces the liquid through the core after the liquid has been forced into the bladder, and the core radially stretches the bladder after the bladder has forced the liquid through the core.

16. The apparatus of claim 15, wherein the core includes a first forked member and further includes a second forked member axially-slidably engaged with the first forked member.

17. The apparatus of claim 16, wherein the bladder defines a first opening and further defines a second opening, the forked member includes an end portion fluid-tightly sealed to the bladder proximal to the first opening, and the second member includes an end portion fluid-tightly sealed to the bladder proximal to the second opening.

18. The apparatus of claim 17, wherein the forked member further includes a pair of elongated branches extending from the end portion of the forked member, and the end portion of the forked member defines a duct including an opening positioned between the branches.

19. The apparatus of claim 18, wherein during the infusion cycle the core radially stretches the bladder by about 12% and the core axially stretches the bladder by about 25%.

20. The apparatus of claim 15, further comprising:
a filter in communication with the core.

21. An apparatus for infusing a pharmaceutically active liquid supplied by an external source, the apparatus comprising:
elastomeric means for pumping the liquid; and
means, coupled to the elastomeric means and at least partially disposed within the elastomeric means, for controlling at least one operation of the elastomeric means.

22. The apparatus of claim 21, further comprising:
a filter in communication with the controlling means
an interchangeable flow regulator in communication with the filter; and
a tamper-resistant housing covering the interchangeable flow regulator.
